(19) United States
(12) Patent Application Publication
(10) Pub. No.: US 2023/0019117 A1
(43) Pub. Date: Jan. 19, 2023

(54) DECENTRALIZED WORKFLOWS FOR SINGLE CELL ANALYSIS

(71) Applicant: FLUENT BIOSCIENCES INC., Watertown, MA (US)

(72) Inventors: Sepehr Kiani, Watertown, MA (US); Ram Santhanam, Watertown, MA (US); Robert Meltzer, Belmont, MA (US); Kristina Fontanez, Arlington, MA (US)

(21) Appl. No.: 17/864,930

(22) Filed: Jul. 14, 2022

Related U.S. Application Data
(60) Provisional application No. 63/222,213, filed on Jul. 15, 2021.

(51) Int. Cl. C12Q 1/6869; C12N 15/10

(52) U.S. Cl. CPC C12Q 1/6869; C12N 15/1065; C12N 15/1096; C12Q 1/6806

(57) ABSTRACT

This disclosure provides a decentralized workflow for analyzing single cell gene expression. The workflow makes use of pre-templated instant partitions to segregate cells into separate compartments to individually capture and barcode RNA from single cells in a massively parallel single tube format. The workflow includes steps for processing the RNA from the single cells for sequencing. Separate portions of the decentralized workflow are performed by a research lab and a core facility, allowing increased flexibility in time and location of protocol steps.

### TECHNICAL FIELD

This disclosure provides methods and reagents for single cell RNA sequencing.

### BACKGROUND

Transcriptional analysis of single cells by RNA sequencing is increasingly recognized as the gold standard for understanding complex cell populations. Single cell RNA sequencing can provide gene expression profiles of single cells and uncover heterogeneity hidden within a sample of different cell phenotypes. As such, methods of single cell RNA sequencing are incorporated into clinical practice to define complex pathological cell populations, e.g., tumors, and characterize their pathogenesis for patient diagnosis and treatment.

For clinics using RNA sequencing, accurate and timely identification of differentially expressed genes is critical for informing on patient health status and treatment monitoring. Although single cell RNA sequencing has the potential to provide those services, the complexity of workflows, high costs of specialized devices, and lack of highly skilled technicians are barriers to its widespread use outside of state-of-the-art laboratories.

The typical single cell RNA sequencing workflow entails sample collection, preparation steps, nucleic acid extraction, cDNA library preparation, PCR amplification, sequencing, and data analysis. Without specialized devices and/or skilled workers, parts of the workflow are inefficient, overly laborious, and error prone. Given that single cell RNA sequencing is a relatively expensive undertaking, and that the results can have a profound impact on patients' lives, extreme caution must be exercised to avoid re-runs and delays, which can be too high a price to pay when patients are waiting for tailored treatments.

### SUMMARY

This invention provides decentralized methods that allow for flexibility in the timing and location of sample processing steps for single cell RNA sequencing. The methods involve a high throughput multi-step workflow with reagents and protocols that are optimized for transporting sample material between discrete workflow steps to different locations for processing. Accordingly, these methods are useful to exploit resources, technical skills, and specialized instrumentation from different facilities to make single cell RNA sequencing more accessible and affordable.

Methods of the invention involve a multi-step process that makes use of pre-templated instant partitions to isolate single cells from bulk samples (many cells) in a single tube format that can be easily transported. The pre-templated instant partitions are useful to segregate large numbers of cells into single cell compartments quickly, and without any expensive instrumentation (e.g., microfluidic devices). As such, samples for single cell RNA sequencing can be initially prepared at almost any location, such as an outdoor research facility or at a remote laboratory. The partitions are formed around hydrogels that template the formation of partitions into stable “reaction chambers” where RNA is prepared for sequencing and/or transport. Methods involve reagents to facilitate sample transport between facilities. For example, some reagents are provided to enhance stability of partitions during transportation. The partitions confine single cells inside individual compartments until sample processing.

The multi-step workflow further involves releasing RNA from the single for capture with barcoded oligos inside partitions. The barcoded oligos are useful for tagging RNA with sequence information corresponding to single cells from which the RNA was released. Advantageously, the barcoded oligos can be attached to the hydrogels by cleavable linkers, thus providing the template particles can serve as reliable vehicles for transporting cell-specific sequences into partitions while also allowing for their easy separation during downstream processes.

Methods of the invention are useful to copy RNA into cDNA, which is more stable than RNA, and thus easier to transport. Methods of the invention can make use of barcoded oligos attached to hydrogels to initiate first-strand cDNA synthesis. Methods may involve polymerization of a cDNA from RNA that is annealed to a 3′ end of the barcoded oligo. Accordingly, copying RNA into cDNA can preserve information from the RNA while simultaneously linking the information to a barcode of the oligo.

In instances where information of mRNA is preserved into barcoded cDNA, methods of the invention are useful for preparing and transporting nucleic acids encoding gene expression of large numbers of single cells all within a single vessel. Advantageously, making those nucleic acids does not require any expensive instrumentation. In fact, making the barcoded cDNA does not even require a thermocycler. Although, in preferred embodiments the cDNA is amplified by, e.g., polymerase chain reaction, into a plurality of stable DNA amplicons that can be stored more effectively under a variety of conditions for safer transport.

In one aspect, this disclosure provides a decentralized method for single cell analysis. The method involves a multi-step workflow (steps (a)-(f)) that begins with (a) partitioning a mixture to generate a plurality of partitions, simultaneously, inside of a vessel, wherein the partitions contain a single cell and a template particle that are isolated from the mixture. The mixture includes an aqueous solution, template particles comprising barcoded oligos, cells, and an oil. The next step involves (b) lysing the cells inside the partitions and capturing mRNA of single cells with barcoded oligos of the template particles. After lysing, the method involves (c) copying the mRNA of the single cells into barcoded cDNA; and then (d) amplifying the barcoded cDNA to create amplicons. The amplicons are useful for (e) preparing sequencing libraries, which are used for (f) sequencing to produce single cell gene expression data. The method is decentralized to take advantage of resources located at a different location than where the sample is collected. That is, the entire multi-step workflow does not occur at the same location. The methods allow researchers to take advantage of specialized core facility equipment and resources, such as technicians that possess skill sets to generate high quality sequencing data. It also allows researchers that may possess qualifications for handling certain sample types, e.g., biohazardous material, to perform initial processing steps that cannot be performed at most sequencing facilities. Accordingly, step (a) is performed by a research lab while step (f) is performed at a core facility.

The partitions are generally generated by vortexing a mixture of cells in an aqueous solution (e.g., media). Vortexing causes the aqueous solution to shear into partitions around each template particle, encapsulating single cells inside droplets with a hydrogel for single cell analysis. In some embodiments, after partitioning, the samples of single cells are transported to a core facility which has resources for performing steps (b), (c), (d), (e), and (f). Since preparing single cells for RNA sequencing analysis does not require any specialized devices, samples for RNA sequencing can be collected and prepared for stable transport from by a research lab at virtually any location, which may be useful for conducting field studies. Generally, transport will involve packaging samples into a container, such as a Styrofoam container, and mailing the container to the core facility. Advantageously, since the sample of cells can be prepared into single cell format within a single vessel, burdens associated with preparing samples for transport to the core facility are minimal and inexpensive.

RNA contained inside single cells is released and captured inside individual partitions with barcoded oligos linked to the hydrogels. According to some embodiments of the invention, after a research lab performs the RNA capture step, method steps (c), (d), (e), and (f), are performed at the core facility. As such, the research lab is able to prepare samples for RNA sequencing analysis even if the lab only has direct access to a heat block (for cell lysis). Because cDNA is more stable than RNA, preferred embodiments involve cDNA synthesis at the research lab. After cDNA synthesis, method steps (d), (e), and (f), are performed at the core facility. Advantageously, cDNA synthesis can be performed at the researching facility without any expensive research devices, in fact, cDNA synthesis does not even require a thermocycler. Accordingly, methods of the invention all expensive analytical instrumentation such as bioanalyzers, thermocyclers, sequencers, qPCR instruments, to be centrally localized, minimizing accessibility barriers to research labs.

In preferred embodiments, a multi-step workflow is performed at a research lab all the way through cDNA amplification. After cDNA amplification, stable cDNA can be easily transported to a core facility, e.g., by mail, where steps (e) and (f) are performed. This workflow format may be desirable as it matches well with instrumentation and resources commonly available at most moderately equipped research labs and core facilities.

Methods of the invention can dramatically reduce time between sample to result. Sequencing RNA is expensive. As such, different samples are often pooled together into a single multiplex sequencing reaction. However, waiting for enough samples to be prepared for a pooled sequencing reaction can prevent investigators from obtaining results within mission critical timeframes. Core facilities often support many different labs and as such are far more efficient for pooling samples and conducting multiplex sequencing reactions. By providing for a decentralized approach to single cell RNA sequencing, these methods facilitate multiplex sequencing for faster turn arounds times between sample to result.

In some instances, the cells prepared for single analysis may contain an infectious agent. Single cell RNA-sequencing of cells containing infectious agents can allow investigations of complex interactions between different host cell types and infections agents, such as a virus or pathogenic bacteria. These investigations may be useful to diagnosis pathogenic infections and/or monitor patient treatment. Sample preparation with cells containing infectious agents is generally carried out at laboratories set up for work and research on easily transmitted pathogens. These laboratories require highly specialized gear and equipment, e.g., protective suits and sealed cabinets, that prevent pathogen transmission. In some instances, these laboratories may be set up at remote locations, for example, at sites of infectious activity, without access to sequencing devices. Core facilities, which generally have sequencing devices, are often not equipped to handle infectious agents. However, by implementing methods of the invention, single cell sequencing data can be produced where a research lab, but not the core facility, is qualified to handle the infectious agent. Methods may involve neutralizing the infectious agent at the research lab before any cell material is transferred to the core facility. Once the infectious agent is neutralized, methods include transporting sample material of the infectious agent to a core facility for sequencing.

Methods of the invention also provide reagents useful for performing certain workflow steps. Some reagents are useful to improve sample integrity during transport. The reagents may be provided by third parties as packaged kits with instructions for use. The kits may be specific to workflow steps performed at the research lab or the core facility. Providing different kits allows investigators to purchase assay-specific supplies, thereby reducing costs and waste associated with kits for carrying out an entire workflow.

### BRIEF DESCRIPTION OF THE DRAWINGS

FIG. 1 illustrates exemplary decentralized multi-step workflows.

FIG. 2 illustrates cDNA synthesis of RNA captured with a template particle.

FIG. 3 shows a kit of the invention.

### DETAILED DESCRIPTION

Cells are the elementary unit of biology, but rarely exist in isolation. More often, biological systems are composed of millions or trillions of cells having somewhat different phenotypes. This complexity can make detection of disease difficult, since analysis of bulk samples can mask the importance of subpopulations. Transcriptional analysis with RNA-seq is useful for understanding the cellular state because it provides genome-wide characterization of RNA expression. When applied to single cells, transcriptome analysis can identify the molecular underpinnings of many biological phenotypes, including the functional properties of tissues, dysregulated gene expression of disease, and detection and/or characterization of a microbe inside a host.

However, single-cell transcriptome sequencing on millions of cells is currently prohibitively expensive—most recognized methods are capable of analyzing just thousands of cells. Recently microfluidic techniques have been shown to detect specific RNA sequences in single cells. Microfluidic techniques are considered high throughput, capable of analyzing thousands (chambers, wells) to millions (microdroplets) of single cells. Unfortunately, microfluidic devices are expensive to use and require costly consumables that limit their application in high throughput field studies. Microfluid devices are also difficult to operate and require frequent maintenance for continued functionality. Accordingly, microfluidic devices are ill-suited for remote, high throughout single cell applications.

This disclosure provides robust single cell RNA sequencing strategies using pre-templated instant partitions to isolate single cells into small volume aqueous droplets inside an immiscible fluid, such as oil. The pre-templated partitions form millions to billions of “nano-lab s” inside a single tube to accommodate high throughput single cell processing in a massively parallel format. The partitions are useful to capture, process, and transport single cells for sequencing. An advantage of partitions is that materials of single cells are confined inside individual compartments preventing its dilution or diffusion until sample processing.

Single cell RNA sequencing is accomplished by decentralized methods that provide for efficient sample to result workflows. The decentralized methods will allow increased flexibility in the timing and location for performing single cell RNA sequencing protocol steps. This flexibility enables new investigative opportunities by facilitating collaborations between researchers at research labs and core facilities, and/or the transfer of samples from high containment laboratories to standard labs after infection agent inactivation.

Methods of the invention distribute workflow steps for single cell RNA sequencing across different geographies. Accordingly, methods of the invention are useful to collect biological samples for examination by RNA sequencing from remote locations where even basic laboratory devices, e.g., thermocyclers, may not be accessible. These methods may be particularly useful in applications in which samples contain an infectious agent. For example, epidemiologists may be deployed to remote locations at a site of a disease outbreak, where sequencing devices are unavailable. Methods of the invention will allow those epidemiologists to rapidly process samples for single cell analysis at a different location equipped with necessary devices, such as a standard next-generation sequencer. As such, these methods are useful for investigators of infectious agents track viral evolution, perform surveillance, manage pandemics, and/or monitor future viral outbreaks from locations having limited resources.

The decentralized methods of the invention enable transfers of sample materials at several district stages of a single cell RNA processing workflow. Methods allow sample materials, including biohazardous materials, to be transported safely, timely, and efficiently from a place where they are collected to a place where they can be analyzed. Methods involve packaging and transporting sample materials, e.g., cells, RNA, or cDNA, in such a way as to protect sample integrity while protecting those engaged in transportation from a risk of infection.

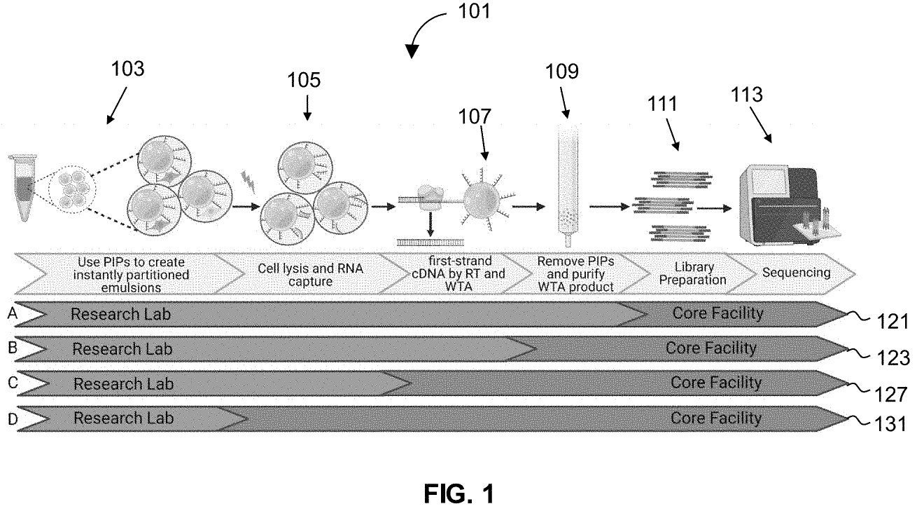

FIG. 1 illustrates exemplary decentralized multi-step workflows 121, 123, 127, 131. The decentralized workflows 121, 123, 127, 131 are based off of a multi-step method 101 useful to prepare libraries large numbers of single cell analysis, for example, of 100 cells, 1,000 cells, 10,000 cells, 100,000,000, 1,000,000 cells, or at least 2,000,000 cells, in a single reaction tube.

The first step, partitioning 103, involves pre-templated instant partitions that partition 103 a mixture and isolate single cells inside compartments for conducting individual, parallel processes. The pre-templated partitions involve template particles, which are generally hydrogel particles that function as templates, causing water-in-oil emulsion droplets to form when mixed inside a mixture of aqueous solution with oil and vortexed or sheared. The template particles may be provided in the aqueous solution (e.g., saline, nutrient broth, water) inside a tube or dried to be rehydrated at time of use. A sample comprising cells may be added into the tube—e.g., directly upon sample collection from a cell culture dish, or after some minimal sample prep step such as centrifuging the cells and re-suspending the cells in a buffered saline solution. Preferably an oil is added to the tube (which will typically initially overlay the aqueous mixture).

For example, an aqueous mixture can be prepared in a reaction tube that includes template particles and cells in aqueous media (e.g., water, saline, buffer, nutrient broth, etc.). The cells can be any cell type that contains RNA. The cells can be obtained from cellular tissue taken from a subject. For example, the cells may be cells taken from a subject by a blood draw. The subject may be suspected of carrying a contagious pathogen. Alternatively, the cells may be tissue culture cells. The cells can be nonadherent or adherent cells, e.g., HeLa cells. The cells can be primary cells, stem cells, epithelial cells, endothelial cells, fibroblast cells, or neurons.

After combining the cells with template particles inside a tube, an oil is added to the tube, and the tube is agitated (e.g., on a vortexer aka vortex mixer). The particles act as template in the formation of monodisperse droplets that each contain one particle in an aqueous droplet, surrounded by the oil. The pre-templated instant partitions are useful to segregate large numbers of cells into single cell compartments quickly, and without any expensive instrumentation (e.g., microfluidic devices). As such, samples for single cell RNA sequencing can be initially prepared at almost any location, such as in the field or at a remote laboratory. The partitions are formed around hydrogels and provide stable reaction chambers that can be transported by courier and/or where RNA is prepared for sequencing.

Preferably, partitioning 103 involves vortexing. Vortexing is preferred for its ability to reliably generate partitions of a uniform size distribution. Uniformity of partitions may be helpful to ensure each “reaction chamber” is provided with substantially equal reagents. Vortexing is also easily controlled (e.g., by controlling time and vortex speed) and thus produces data that are more easily reproduceable. Vortexing may be performed with a standard bench-top vortexer or a vortexing device as described in co-owned U.S. patent application Ser. No. 17/146,768, which is incorporated by reference.

However, for applications in which samples are processed at a remote location without access to a vortexer, partitioning 103 may involve agitating the tube containing the mixture using any other method of controlled or uncontrolled agitation, such as shaking, pipetting, pumping, tapping, and the like. After agitating (e.g., vortexing), a plurality (e.g., thousands, tens of thousands, hundreds of thousands, one million, two million, ten million, or more) of aqueous partitions is formed essentially simultaneously inside the tube. Vortexing causes the fluids to partition into a plurality of monodisperse droplets. A substantial portion of droplets will contain a single template particle and a single cell. Droplets containing more than one or none of a template particle or target cell can be removed, destroyed, or otherwise ignored.

The next step of the method 101 involves lysing 120 the single cells and capturing released RNA inside the partitions. Cell lysis may be induced by a stimulus, such as, for example, lytic reagents, detergents, or enzymes. Reagents to induce cell lysis may be released by the template particles from internal compartments. In some embodiments, lysing involves heating the droplets to a temperature sufficient to release lytic reagents contained inside the template particles into the monodisperse droplets. In some embodiments, lysing may involve heating the partitions to a temperature sufficient to release lytic reagents, such as, divalent cations, contained inside the hydrogels into the partitions. Ly sing may be accomplished using mechanical, chemical, or enzymatic means, the addition of heat, divalent cations (e.g., Mn2+ and/or Mg2+), or any combination thereof.

Upon cell lysis, RNA and other cellular contents (e.g., DNA) are released from the cells and into the partitions for capture with the barcoded oligos provided attached to the template particles. The oligos include unique barcodes specific to each template particle. Accordingly, upon capture, i.e., hybridization of complementary base pairs, of the RNA with respective complementary portions of oligos (e.g., poly-T sequences, RNA of single cells is effectively linked by a common barcode sequence. Since each partition includes only one single cell and one template particle, the unique barcode sequences of any one template particles is useful for indexing RNA with information linking it to the single cell from which it was derived.

Once cell lysis and RNA capture has been performed 120 has been performed, the method 101 involves first-stand cDNA synthesis. For background overview, see generally Gubler, 1983, A simple and very efficient method for generating cDNA libraries, Gene 25(2-3):263-9 and Figueiredo, 2007, Cost effective method for construction of high quality cDNA libraries, Biomolecular Eng 24:419-421, both incorporated by reference.

In preferred embodiments, the barcoded oligos are used to initiate first-strand cDNA synthesis. That is, polymerization of the first-strand of cDNA can be initiated off the free ends of the barcoded oligos, thereby producing a cDNA molecular, linked to a template particle, which captures all single cell information that is encoded by the captured RNA. Moreover, because the information of RNA is preserved into barcoded cDNA, methods of the invention are useful for preparing and transporting nucleic acids encoding gene expression of large numbers of single cells all within a single vessel. Advantageously, making those nucleic acids does not require any expensive instrumentation. In fact, making the barcoded cDNA does not even require a thermocycler. Although, in some preferred embodiments, the cDNA is amplified by, e.g., polymerase chain reaction, into a plurality of stable DNA amplicons that can be stored more effectively under a variety of conditions for safer transport.

Preferably, one or a plurality of the partitions will each have a plurality of cDNA that include droplet-specific oligonucleotide barcodes for a plurality of corresponding RNA that were partitioned into the droplets by the partitioning 103. Forming the cDNA may include attaching amplification primer-binding sites (such as first and second universal priming sequences at the ends of the cDNAs), and the method 101 optionally includes amplifying (before transfer) the cDNA into amplicons, which may be stored, transported, or processed further into sequencing libraries. For example, the amplicons may be processed for sequencing using a sequencer such as a next-generation sequencing (NGS) instrument.

Next, the barcoded cDNA is separated from the template particles and amplified 109 to produced amplicons for sequencing analysis. During first-strand cDNA synthesis, a reverse transcriptase binds and initiates synthesis of cDNA of the RNA, which is connected to the template particle non-covalently, by Watson-Crick base-pairing. The cDNA that is synthesized is covalent linked to the particle by virtue of the phosphodiester bonds formed by the reverse transcriptase. Before amplification, the template particle is separated from the synthesized cDNA.

The cDNA, together with the oligo to which it is covalently linked, can be released from the template particle in a controlled fashion using a USER enzyme. Addition of the USER enzyme is helpful to cleave integrated uracil bases of the oligo-template particle linker, thereby releasing the cDNA. The released cDNA molecules can be transferred to a different facility for second-strand synthesis.

The cDNA molecules are preferably amplified 109 by whole transcriptome amplification, which is useful for comprehensively characterizing global transcriptome activity of single cells. Advantageously, whole transcriptome amplification amplifies the transcriptome, even in the face of low starting material and/or when samples are heavily degraded due to insufficient preservation.

Whole transcriptome amplification reagents and protocols can be obtained from commercially available kits, such as, the RNA amplification kit sold under the trade name Rapid Amplification of Total RNA, by Sigma. The amplicon products from whole transcriptome amplification reactions are divided into two size specific populations, from which sequencing libraries are prepared preparation.

Some embodiments may employ a single primer isothermal amplification (SPIA) method to amplify the cDNA. Amplified cDNA can then purified using a column, such as with the purification kit sold under the trade name MinElute Reaction Cleanup Kit (Qiagen; Valencia, Calif., USA), according to manufacturer's protocol. For further discussion on methods of whole transcriptome amplification see, Faherty, 2015, Evaluating whole transcriptome amplification for gene profiling experiments using RNA-Seq, BMC Biotechnology 15(65), incorporated by reference. Alternatively, to reduce sequencing expenses, methods of the invention may involve selective amplification of RNA of associated with specific genes of interest. The genes of interest may be amplified by PCR amplification using gene specific primers. To select genes for targeted amplification, investigators may research existing gene expression databases, for example, Gene, or the Gene Expression Omnibus database, which are freely available on the web by National Center for Biotechnology Information, to identify genes associated with a disease or condition of interest. Making the primers can be performed in a lab using methods known in the art, or the primers can be purchased from a third party

Next, the method involves preparing 111 a sequencing library. The libraries are preferably prepared at a core facility. The sequencing library may be prepared by amplifying the amplicons using primers that provide sequencing adapters, e.g., adapters compatible with an Illumina sequencing instrument, to resultant PCR products. For discussion, see Head, 2018, Library construction for next-generation sequencing: Overviews and challenges, Biotechnique 56(2), incorporated by reference. It is contemplated that the P5 sequences, the P7 sequence, and the index segment may be the sequences use in NGS indexed sequences such as performed on an NGS instrument sold under the trademark ILLUMINA, and as described in Bowman, 2013, Multiplexed Illumina sequencing libraries from picogram quantities of DNA, BMC Genomics 14:466, incorporated by reference. A hexamer priming method may be used. The hexamer segments may be random hexamers or selective hexamers (aka not-so-random hexamers). Some embodiments may make use of not-so-random (NSR) oligomers (NSROs). See Armour, 2009, Digital transcriptome profiling using selective hexamer priming for cDNA synthesis, Nat Meth 6(9):647-650, incorporated by reference. Preferably, the particles are linked to capture oligos that include one or more primer binding sequences P5, P7 cognate to PCR primers that may be used in an option downstream amplifying step (such as PCR or bridge amplification).

The sequencing libraries are then sequenced 113, preferably using a next-generation sequencer. Although sequencing 113 the libraires may be performed by methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid molecule sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, U.S. Pat. Nos. 6,828,100, 6,833,246, and 6,911,345, each incorporated by reference. After sequencing, the sequencing data may be subsequently processed for analysis.

The method 101 involves a multi-step process that can advantageously be paused at various steps, such as, after cell capture, after cell lysis and mRNA capture, after first strand cDNA synthesis, and after whole transcriptome cDNA amplification (WTA). Accordingly, the method 101 provides a useful format for researchers to being library preparation at a first location (e.g., a research lab) and then transfer the samples to a separate facility (e.g., a core facility) to complete the library preparation and sequencing processes. There are several advantages to this split workflow configuration, for example, front end processes may be performed in any cell or molecular biology lab while downstream processes are performed at well-equipped core facilities. This is useful to optimize timing of cell isolation to stable transcriptome capture, while reducing hands-on time for the endpoint users. Moreover, these methods are also useful for minimizing the purchase and storage of materials at a point of collection and allows core facilities, e.g. at collection points, to bundle multiple samples from multiple users for efficient library processing. Accordingly, some embodiments provide for large panels of unique oligo indexes, which allow for easy sample pooling at a central facility, where experienced operators for sequencing library preparation and quantification, minimizing risk of poor assay performance.

The multi-step workflows provided by the method 101 can be initiated at a first location, e.g., by a research lab, and completed at a second location, e.g., a core facility. The research lab may be at a facility site that provides controlled conditions in which scientific or technological research, experiments, and measurement may be performed. The research lab may have access to basic molecular biology equipment, such as vortexers, heath baths, heat blocks, thermocyclers. The research lab is not limited by a geographical location. The research lab may be at a physicians' offices, a clinic, a hospital, etc. The research lab can be at an outdoor research site. For example, the research lab may be involved in a field research study, collecting samples to produce quantitative data useful to understand a natural environment or monitor pathogenic transmissions.

Core facilities encompass institutions designed to support scientific researchers with specialized expertise, service, and access to advanced instrumentation. In general core facilities consist of dedicated space, specialized scientific equipment, and expert staff. The guiding principle behind core facilities is that through the efforts of dedicated professional scientists, managers, and administrators, shared research platforms ensure more efficient resource utilization, as well as specialist instruction, support, and management. Thus, core facilities can take many forms ranging from individual pieces of shared research equipment to large multi-component research centers. All types of research institutions and universities, as well as pharmaceutical and biotech companies, can incorporate a core facility concept as an efficient and cost-effective way to leverage research expertise and specialized instrumentation, and ensure appropriate technical and operational oversight. Core facility staff are generally scientists who are skilled in aspects of data acquisition and analysis. The core facility is different than a research lab in that it includes advanced or specialized instrumentation (e.g., sequencers) that are too expensive to be efficiently employed by a research lab.

According to one embodiment, the decentralized workflow 121 involves initiating the method 101 at a research lab by partitioning 103 a sample of cells and processing the sample through whole transcriptome amplification 109. Sequencing library preparation 111 and sequencing 113 are performed at a core facility. Advantageously, this embodiment matches sample processing steps to materials and skillsets that are often found at research labs and core facilities. For example, research labs generally have access to vortexers for partitioning 103 cell samples. Research labs generally have access to heat blocks which are useful to lyse 105 cells and capture RNA onto template particles. Research labs generally have access to thermocyclers for cDNA synthesis and whole transcriptome amplification. Moreover, the products or whole transcriptome amplification (DNA) are highly stable. Making the transfer of these products to a core facility relatively easy. For example, the DNA may even be transferred at room temperature, which is cost-effective and easy. For example, the amplified sample may be directly dried onto a paper matrix. The options for paper matrices include a variety of untreated matrices (e.g., Guthrie or Whatman 903 cards) and chemically treated matrices (i.e., Whatman FTA technology). Alternatively, the samples, cab be shipped in a tube, such as, an Eppendorf tube, in a solution of water or saline. Stabilization agents, such as EDTA, may be added to the solution to enhance nucleic acid stability during transport.

Samples (e.g., cells, RNA, cDNA) can be transferred between a research lab and a core facility by courier or mail. For example, samples can be shipped refrigerated in dry shippers. These are insulated packages containing refrigerated liquid nitrogen fully absorbed in a porous filter within the shipper. The samples can be shipped inside Eppendorf tubes packaged in a leak-proof container containing dry ice, e.g., a Styrofoam box. The samples can be mailed through a postal service.

According to a different embodiment, a decentralized workflow 123 involves preparing samples for RNA sequencing a research lab up and through first-strand cDNA synthesis 107.

After first-strand cDNA synthesis, stable DNA transcripts, which may be bound to the template particles, can be transferred to a core facility where whole transcriptome sequencing is performed. Advantageously, this method obvious the need for a thermocycler at the research lab. The cDNA can be transferred to the core facility inside a sample preparation tube on dry ice. The cDNA may be preserved in a solution comprising EDTA.

In other embodiments, a decentralized workflow 127 involves method steps for cell partitioning 103, cell lysis 105 and RNA capture at a research lab. After RNA capture, samples are transported to a core facility for cDNA synthesis 107, amplification 109, library preparation 111 and sequencing 113. Advantageously, this embodiment requires very few materials of a research lab, e.g., no thermocycler, sequencer, etc.). Moreover, some embodiments can make use of reagents of the invention that facilitate transfer of the captured RNA to the core facility. For example, reagents include a cell lysis buffer which can be used during cell lysis 105 to lyse the cells. The cell lysis buffer can include multiple modalities of DNAses and RNAses inactivation to preserve RNA integrity during transport. Reagents can prevent proteolytic activity by the incorporation of Protease K, to digest protein and remove contaminants. Methods can prevent disulfide bond hydrolysis by the addition of dithiothreitol DTT.

In a different embodiment, a decentralized workflow 131 involves partitioning 103 a sample of cells at a research facility. The tube containing the partitioned sample is then transported to the core facility for further processing (i.e., performing steps 105-111) and subsequent sequencing 113. Since this embodiment of preparing single cells for RNA sequencing analysis at the research lab does not require any specialized devices, samples for RNA sequencing can be collected and prepared for stable transport by the research lab form virtually any location, which may be useful for conducting field studies. Generally, transport will involve packaging the tube of single cell partitions into a container, such as a Styrofoam container, and mailing the container to the core facility. To facilitate transport, methods of the invention may involve adding surfactants, discussed below, to the oil mixture which is used during the partitioning 103 step described above. The surfactants are useful to stabilize the partitions during transports, thereby ensuring single cells are kept in isolation. Advantageously, since the sample of cells can be prepared into single cell format within a single vessel, burdens associated with packaging samples for transport to the core facility are minimal and inexpensive. Preferably, the tube is put on ice to prevent any further cell activity, e.g., transcription, from occurring after sample collection.

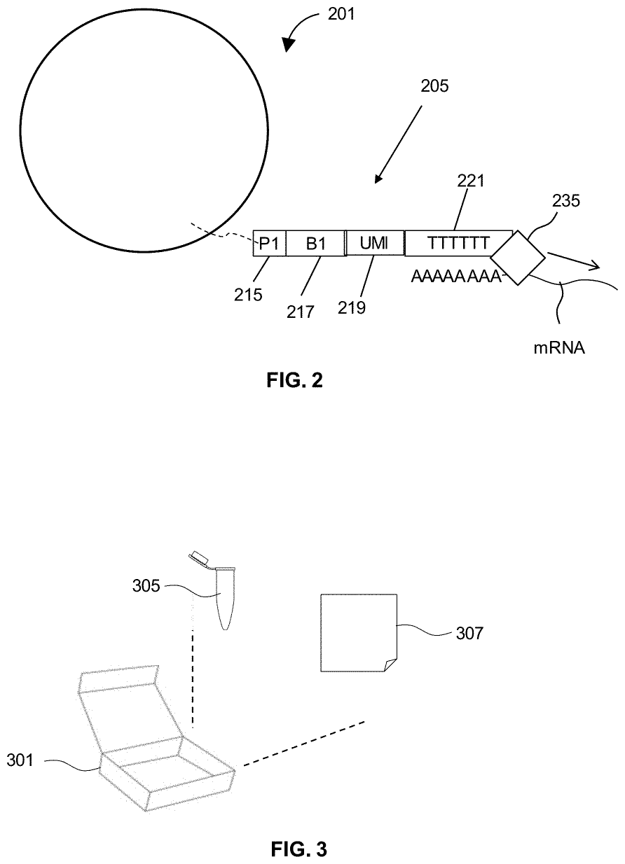

FIG. 2 illustrates cDNA synthesis of RNA captured with a template particle 201. The RNA (mRNA) is captured by the template particle 201 by hybridization with a barcoded oligo 205. For simplicity, only a single barcoded oligo 205 is illustrated. In practice, any number of barcoded oligos may be attached. For example, the template particle 201 may be decorated with millions or billions or more oligos.

The capture of the RNA takes place inside a pre-templated instant partition (not shown), following lysis of a single cell as described herein. The template particle 201 and oligo 205 can be linked by a cleavable bond, e.g., a protease cleavable peptide. The oligo 205 can include, from 5′ to 3′, a PCR primer binding site 215, a barcode sequence 217, a unique molecular identifier (UMI) 219, and a capture sequence 221.

After capture (i.e., oligo-RNA hybridization), cDNA synthesis of the captured RNA can be performed with reverse transcriptase 235. During cDNA synthesis, the reverse transcriptase 235 creates a copy of the RNA molecule into cDNA that that is covalently attached to the barcode by way of the synthesized phosphodiester bonds. The barcode sequence 217 encodes a sequence of nucleotides that is unique to the template particle 201. Since each single cell is captured with a different template particle, barcode sequence 217 allows every RNA sequence read of a single common cell to be grouped together. As such, sequence reads of RNA captured by template particles are useful to analyze expression of single cells in bulk.

Methods of the invention are useful to study RNA of single cells. Methods of the invention provide involve several distinct strategies for which the primary objective of an RNA sequencing experiment will need to be considered before making a decision on a best library protocol. For example, if the objective is discovery of complex and global transcriptional events, the library should capture the entire transcriptome, including coding, noncoding, anti-sense and intergenic RNAs, with as much integrity as possible. However, other embodiments, the objective may be study only the coding mRNA transcripts that are translated into the proteins, which can be captured with poly-T capture oligos. Yet embodiments may involve preparations of RNA sequencing libraries to study only small RNA, most commonly miRNA, but also small nucleolar RNA (snoRNA), piwi-interacting RNA (piRNA), small nuclear RNA (snRNA), and transfer RNA (tRNA). Advantageously, methods of the invention are useful to capture single cells, together with all of its corresponding RNA species, inside stable pre-templated instant partitions, which can be transported to distant locations for library preparation. An advantage of partitions is that materials of single cells are confined inside individual compartments preventing its dilution or diffusion until sample processing.

Methods of the invention are useful for sequencing RNA from single cells. The RNA sequencing data provides informs on a single cells transcriptome, whose expression correlates well with cellular traits and changes in cellular state. For example, at any moment each cell makes mRNA from only a fraction of the genes it carries. If a gene is used to produce mRNA, it is considered “on”, otherwise it is considered “off”. Gene expression profiling may include measuring the relative amount of mRNA expressed in two or more conditions. For example, cells may be modified by an RNA guide that is thought to produce an “on” switch in a gene, an RNA guide that is thought to produce as “off” switch in a gene, and an RNA guide that is thought to produce no change in the gene. The gene expression profile provides information as to what the changes made by the guide RNAs in DNA actually result in phenotypically in the cell. Gene expression profiling may also provide information as the editing capacity of RNA guides, for example when multiple RNA guides targeting the same “on” switch are analyzed in parallel to assess varying levels of gene expression level changes.

Accordingly, methods of the invention may involve generating expression profiles of the single cell RNA sequencing data. In some embodiments, gene expression profiles may be made using analysis tools openly available, such as, TopHat2 (Johns Hopkins University for Computational Biology), Cufflinks (University of Washington, Cole Trapnell Lab), and DESeq2 (See Love MI, Huber W and Anders S, 2014, Moderated estimation of fold change and dispersion for RNA-seq data with DESeq2, Genome Biology, 15, pp. 550, incorporated herein by reference) may be used to align RNA sequences and to determine expression levels and identify differential expression corresponding with cell types. Expression levels may be normalized to expression levels of a housekeeping gene or other control measured in the sample. For example, the normalized expression levels may be compared to a threshold expression level from a single cell of a known cell state

FIG. 3 shows a kit 301 of the invention. The kit 301 can include reagents for performing methods described herein. The kit 301 can be designed to accomplish specific steps workflow steps of the method 101 described above. For example, making reference to FIG. 1, the kit 301 may be designed to accomplish any one of decentralized workflows (i.e., 121, 123, 127, 131) performed at the research lab or the core facility. Providing different kits allows investigators to purchase assay-specific supplies, thereby reducing costs and waste associated with kits for carrying out an entire workflow. For example, the kit 301 may include a tube 305 containing template particles. The template particles may be provided in an aqueous media (e.g., saline, nutrient broth, water) or dried to be rehydrated at time of use. Preferably, the template particles are decorated with oligos for the capture of RNA (e.g., mRNA). The oligos can be designed to promote stable and specific annealing with poly-A tails of mRNA. Preferably, the oligos include UMIs. The UMIs can be designed with unique sequences that discourage non-specific binding the mRNA. For example, the UMIs can be designed to have low thymine levels, and preferably few instances of repeated thymine nucleotides. The kit can contain instructions 309 for carrying out workflow steps. The kit 301 can include reagents that facilitate transport of sample material between facilitates (e.g., a research lab and a core facilitate). For example, the kit 301 may include reagents for stabilizing nucleic acids during transfer. The kit 301 may include reagents that stabilize emulsions or enhance thermostability. For example, the kit 301 can include certain surfactant molecules, e.g., polymers, proteins, or particles that assemble at an interface between an oil and aqueous solution to prevent liquids from separating.

Suitable surfactants for may include, for example, Ran or ionic Krytox. The surfactant may be a PEG—PFPE amphiphilic block copolymer surfactant, for example, as discussed in Hatori, 2018, Particle-templated emulsification for microfluidics-free digital biology, Anal Chem 90:9813-9820, incorporated by reference.

In some embodiments, the kit 301 is made to order. For example, an investigator may use, e.g., an online tool to select from a list of decentralized workflows (i.e., 121, 123, 127, 131) described above. The investigator may use the online search tool to identify whether the kit is for use at a research lab or at a core facility. Different kits can be made pending on the workflow being performed. For example, some kits 301 only include reagents for partitioning samples. The kit 301 can include template particles, cell suspension buffer, cell dilution buffer, and a partitioning reagent. The partitioning reagent may comprise an oil and surfactant. The oil is preferably a fluorinated oil.

In some instances, the kit 301 will also include reagents for cell lysis and RNA capture. The kit 301 may include a breaking buffer, a wash buffer, and a de-partitioning reagent. The kit may include reagents for inactivating ribonucleases, such as those described in U.S. Pat. No. 6,777,210, which is incorporated by reference. The kit 301 can also include reagents for making a first strand of cDNA. For example, the kit 301 may include reverse transcriptase, and dNTPs. The kit can include reagents for cDNA amplification. The kit can include USER enzyme. In some instances, a kit 301 will include reagents for generating a sequencing library. For example, the kit 301 may include indexes for sample multiplexing.

The template particles may provide oligonucleotides for target capture and barcoding of polyadenylated RNA. Barcodes specific to each template particle may be any group of nucleotides or oligonucleotide sequences that are distinguishable from other barcodes within the group. Accordingly, a partition encapsulating a template particle and a single cell provides to each nucleic acid molecule released from the single cell the same barcode from the group of barcodes. The barcodes provided by template particles are unique to that template particle and distinguishable from the barcodes provided to nucleic acid molecules by every other template particle. Once sequenced, by using the barcode sequence, the nucleic acid molecules can be traced back to the single cell based on the barcode provided by the template particle that the single cell was partitioned with. Barcodes may be of any suitable length sufficient to distinguish the barcode from other barcodes. For example, a barcode may have a length of 4, 5, 6, 7, 8, 9, 10, 11, 12, 13, 14, 15, 16, 17, 18, 19, 20, 21, 22, 23, 24, 25 nucleotides, or more.

The barcodes unique to each template particle may be pre-defined, degenerate, and/or selected at random. Barcodes may be added to nucleic acid molecules by “tagging” the nucleic acid molecules with the barcode. Tagging may be performed using any known method for barcode addition, for example direct ligation of barcodes to one or more of the ends of each nucleic acid molecule. Nucleic acid molecules may, for example, be end repaired in order to allow for direct or blunt-ended ligation of the barcodes. Barcodes may also be added to nucleic acid molecules through first or second strand synthesis, for example using capture probes, as described herein below.

In some methods of the invention, an index or barcode sequence may comprise unique molecule identifiers (UMIs). UMIs are a type of barcode that may be provided to a sample to make each nucleic acid molecule, together with its barcode, unique, or nearly unique. This may be accomplished by adding one or more UMIs to one or more capture probes of the present invention. By selecting an appropriate number of UMIs, every nucleic acid molecule in the sample, together with its UMI, will be unique or nearly unique.

UMIs are advantageous in that they can be used to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, Counting Molecules in cell-free DNA and single cells RNA″, Karolinska Institutet, Stockholm Sweden, incorporated herein by reference.

In some embodiments of the template particles, a variation in diameter or largest dimension of the template particles such that at least 50% or more, e.g., 60% or more, 70% or more, 80% or more, 90% or more, 95% or more, or 99% or more of the template particles vary in diameter or largest dimension by less than a factor of 10, e.g., less than a factor of 5, less than a factor of 4, less than a factor of 3, less than a factor of 2, less than a factor of 1.5, less than a factor of 1.4, less than a factor of 1.3, less than a factor of 1.2, less than a factor of 1.1, less than a factor of 1.05, or less than a factor of 1.01.

Template particles may be porous or nonporous. In any suitable embodiment herein, template particles may include microcompartments (also referred to herein as “internal compartment”), which may contain additional components and/or reagents, e.g., additional components and/or reagents that may be releasable into monodisperse droplets as described herein. Template particles may include a polymer, e.g., a hydrogel. Template particles generally range from about 0.1 to about 1000 μm in diameter or larger dimension. In some embodiments, template particles have a diameter or largest dimension of about 1.0 μm to 1000 μm, inclusive, such as 1.0 μm to 750 μm, 1.0 μm to 500 μm, 1.0 μm to 250 μm, 1.0 μm to 200 μm, 1.0 μm to 150 μm 1.0 μm to 100 μm, 1.0 μm to 10 μm, or 1.0 μm to 5 μm, inclusive. In some embodiments, template particles have a diameter or largest dimension of about 10 μm to about 200 μm, e.g., about 10 μm to about 150 μm, about 10 μm to about 125 μm, or about 100 μm to about 100 μm.

In practicing the methods as described herein, the composition and nature of the template particles may vary. For instance, in certain aspects, the template particles may be microgel particles that are micron-scale spheres of gel matrix. In some embodiments, the microgels are composed of a hydrophilic polymer that is soluble in water, including alginate or agarose. In other embodiments, the microgels are composed of a lipophilic microgel. In other aspects, the template particles may be a hydrogel. In certain embodiments, the hydrogel is selected from naturally derived materials, synthetically derived materials and combinations thereof. Examples of hydrogels include, but are not limited to, collagen, hyaluronan, chitosan, fibrin, gelatin, alginate, agarose, chondroitin sulfate, polyacrylamide, polyethylene glycol (PEG), polyvinyl alcohol (PVA), acrylamide/bisacrylamide copolymer matrix, polyacrylamide/poly(acrylic acid) (PAA), hydroxyethyl methacrylate (HEMA), poly N-isopropylacrylamide (NIPAM), and polyanhydrides, poly(propylene fumarate) (PPF).

In some embodiments, the presently disclosed template particles further comprise materials which provide the template particles with a positive surface charge, or an increased positive surface charge. Such materials may be without limitation poly-lysine or Polyethyleneimine, or combinations thereof. This may increase the chances of association between the template particle and, for example, a cell which generally have a mostly negatively charged membrane.

Other strategies may be used to increase the chances of templet particle-target cell association, which include creation of specific template particle geometry. For example, in some embodiments, the template particles may have a general spherical shape but the shape may contain features such as flat surfaces, craters, grooves, protrusions, and other irregularities in the spherical shape.

Any one of the above described strategies and methods, or combinations thereof may be used in the practice of the presently disclosed template particles and method for targeted library preparation thereof. Methods for generation of template particles, and template particles-based encapsulations, were described in International Patent Publication WO 2019/139650, which is incorporated herein by reference.

### INCORPORATION BY REFERENCE

References and citations to other documents, such as patents, patent applications, patent publications, journals, books, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. A decentralized method for single cell analysis, the method comprising steps:
(a) partitioning a mixture to generate a plurality of partitions, simultaneously, inside of a vessel, wherein the partitions contain a single cell and a template particle that are isolated from the mixture comprising:
an aqueous solution;
template particles comprising barcoded oligos;
cells; and
an oil;
(b) lysing the cells inside the partitions and capturing mRNA of single cells with barcoded oligos of the template particles;
(c) copying the mRNA of the single cells into barcoded cDNA;
(d) amplifying the barcoded cDNA to create amplicons;
(e) preparing sequencing libraries from the amplicons;
(f) sequencing the libraries to produce single cell gene expression data;
wherein, step (a) is performed at a research lab and step (f) is performed at a core facility.

2. The method of claim 1, wherein steps (e) and (f) are performed at the core facility.

3. The method of claim 1, wherein steps (d), (e), and (f), are performed at the core facility.

4. The method of claim 1, wherein steps (c), (d), (e), and (f), are performed at the core facility.

5. The method of claim 1, wherein steps (b), (c), (d), (e), and (f), are performed at the core facility.

6. The method of claim 1, wherein partitioning comprises vortexing the mixture to shear the aqueous solution into droplets surrounded by the oil.

7. The method of claim 6, wherein the droplets are formed around the template particles.

8. The method of claim 6, wherein the oil comprises a surfactant that stabilizes the partitions.

9. The method of claim 8, further comprising mailing the vessel comprising the partitions to the core facility for processing.

10. The method of claim 1, wherein the cDNA is linked to the template particles via the barcoded oligos.

11. The method of claim 10, wherein the cDNA is mailed to the core facility.

12. The method of claim 11, wherein no PCR steps are performed by the research lab.

13. The method of claim 1, wherein the amplicons are generated by the research lab.

14. The method of claim 13, wherein the amplicons are mailed to the core facility.

15. The method of claim 1, wherein at least one of the steps is performed by the research lab at a remote location.

16. The method of claim 1, wherein the cells comprise an infectious agent.

17. The method of claim 16, wherein the research lab, but not the core facility, is qualified to handle the infectious agent.

18. The method of claim 17, wherein the infectious agent is neutralized by the research lab before any cell material is transferred to the core facility.

19. The method of claim 1, wherein the research lab and the core facility perform the method steps with a sample preparation kit comprising reagents and instructions that is provided by a third party.

20. The method of claim 19, wherein the kits are specific to the steps performed at the research lab and core facility.
